(19) United States
(12) Patent Application Publication
(10) Pub. No.: US 2009/0298950 A1
(43) Pub. Date: Dec. 3, 2009

(54) CYCLOPENTANE/CYCLOPENTENE ALDEHYDE OR KETONE DERIVATIVES AND THEIR USE AS ODORANTS

(76) Inventors: Jerzy A. Bajgrowicz, Zurich (CH); Urs Mueller, Duebendorf (CH)

Correspondence Address: NORRIS, MCLAUGHLIN & MARCUS, 875 THIRD AVE, 18TH FLOOR, NEW YORK, NY, 10022, US

(21) Appl. No.: 12/376,010

(22) PCT Filed: Aug. 7, 2007
(86) PCT No.: PCT/CH07/00384

§ 371 Date: Mar. 24, 2009

(30) Foreign Application Priority Data
Aug. 11, 2006 (GB) 0616002.2

(51) Int. Cl. A61K 31/12; C07C 49/11; C07C 47/11; A61K 31/11; A61Q 13/00; A61K 8/34; A61K 8/35; C11D 3/50

(52) U.S. Cl. 514/690; 568/379; 568/420; 514/693; 512/8; 510/102

(57) ABSTRACT

The present invention relates to campholytic aldehyde condensates of formula (I)

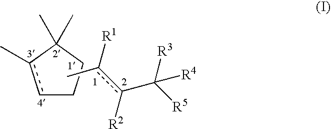

wherein R¹-R⁵ have the same meaning as given in the specification.

It furthermore relates to a method of their production and to fragrance compositions and fragrance applications comprising them.

The present invention refers to a novel class of campholytic aldehyde condensates having useful woody-ionone-like odor notes, and to their use as odorants. This invention relates furthermore to a method for their production and to fragrance compositions comprising them.

In the fragrance industry there is a constant demand for new compounds that enhance, modify or improve on odor notes.

The prior art reports several derivatives of campholenic aldehyde. Two well-known compounds which have appeared on the market are Ebanol® and Polysantol®, both of which possess sandalwood odor notes, and a real sandalwood character can be obtained throughout the whole perfume in which they are incorporated in top, middle and base notes.

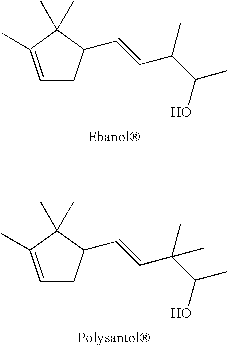

Up to now it has not been possible to draw a complete correlation between structure and odour, and it has not therefore generally been possible to predict which compounds will possess a useful or pleasing odour, or what the particular odour description of any given compound will be.

Surprisingly, it has now been found that by shortening the spacer group between the osmophoric center, namely the oxygen atom, and the cyclic ring system, i.e. the lipophilic part of the molecule, by one carbon atom, there may be obtained a novel class of compounds possessing floral, woody, lemon-type, fruity (raspberry), anis odours, almost completely lacking the distinct sandalwood aspects of the prior art compounds. Ebanol® and Polysantol® are the known odorant molecules having the closest structure to the compounds of the present invention. Whereas the odour of Ebanol® and Polysantol® has a creamy, woody and slightly urinaceous, animalic tonality typical of East Indian Sandalwood (Santalum album L.) oil, the compounds of the present invention as hereinbelow described have a distinct ionone-type, warm woody, balsamic, floral odour of the deep sweetness reminiscent of that of violet flowers and a fruity connotation resembling raspberries.

Accordingly the present invention refers in one of its aspects to the use as flavour or fragrance ingredient of a compound of formula (I)

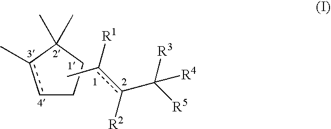

wherein
R¹ and R² are independently hydrogen, or C₁-C₃ alkyl, e.g. ethyl;
C-1 is attached to C-1′ or C-4′;
the dotted line between C-1 and C-2 represents together with the carbon-carbon bond a double bond or a single bond;
the dotted line between C-3′ and C-4′ represents together with the carbon-carbon bond a double bond or a single bond;
I) R³ and R⁴ together with the carbon atom to which they are attached form a carbonyl group; and
   R⁵ is hydrogen, C₁-C₆ alkyl, e.g. ethyl, butyl, or isopropyl, or C₂-C₆ alkenyl, e.g. butenyl or isopropenyl;
   or
II) R³ is hydroxyl; and
   R⁴ and R⁵ are independently from each other hydrogen, C₁-C₆ alkyl, or C₂-C₆ alkenyl; with the proviso that at least one of R¹, R², R⁴ and R⁵ is not hydrogen;
and the compound of formula (I) comprises up to 20 carbon atoms, preferably 11 to 18, e.g. 12, 13, 14, or 15.

The compounds of formula (I) may comprise several chiral centres and as such may exist as a mixture of stereoisomers, or they may be resolved as isomerically pure forms. Resolving stereoisomers adds to the complexity of manufacture and purification of these compounds and so it is preferred to use the compounds as mixtures of their stereoisomers simply for economic reasons. However, if it is desired to prepare individual stereoisomers, this may be achieved according to methods known in the art, e.g. preparative HPLC and GC, crystallization or by departing from chiral starting materials, e.g. starting from enantiomerically pure or enriched raw materials such as terpenoids, and/or by applying stereoselective synthesis.

In particular embodiments are compounds of formula (I) wherein the relative configuration of the ring system is 1′R or 1′S, as shown by formula (Ia) and (Ib).

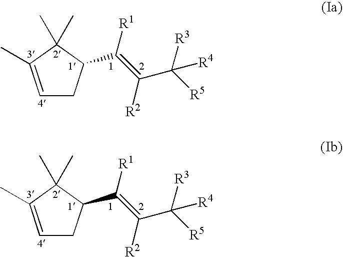

The double bond being either E or Z.

Particular preferred compounds of formula (I) are 4-(2,2,3-trimethylcyclopent-3-enyl)-but-3-en-2-one, (3E)-3-methyl-4-(2,3,3-trimethylcyclopent-1-enyl)but-3-en-2-one and (3E)-3-methyl-4-(2,3,3-trimethylcyclopent-1-enyl)but-3-en-2-ol.

The compounds according to the present invention may be used alone or in combination with known odorant molecules selected from the extensive range of natural and synthetic molecules currently available, such as essential oils and extracts, alcohols, aldehydes and ketones, ethers and acetals, esters and lactones, macrocycles and heterocycles, and/or in admixture with one or more ingredients or excipients conventionally used in conjunction with odorants in fragrance compositions, for example, carrier materials, and other auxiliary agents commonly used in the art, e.g., solvents such as dipropylen glycol, isopropylmyristate, and triethylcitrate.

The following list comprises examples of known odorant molecules, which may be combined with the compounds of the present invention:
essential oils and extracts, e.g. oak moss absolute, basil oil, tropical fruit oils, such as bergamot oil and mandarine oil, mastic absolute, myrtle oil, palmarosa oil, patchouli oil, petitgrain oil, wormwood oil, lavender oil, rose oil, jasmin oil, ylang-ylang oil and sandalwood oil.
alcohols, e.g. cis-3-hexenol, cinnamic alcohol, citronellol, Ebanol™, eugenol, farnesol, geraniol, menthol, nerol, rhodinol, Super Muguet™, linalool, phenylethyl alcohol, Sandalore™, terpineol and Timberol™ (1-(2,2,6-Trimethylcyclohexyl)hexan-3-ol).
aldehydes and ketones, e.g. citral, hydroxycitronellal, Lilial®, methylnonylacetaldehyde, anisaldehyde, allylionone, verbenone, nootkatone, geranylacetone, α-amylcinnamic aldehyde, Georgywood™, hydroxycitronellal, iso E Super®, Isoraldeine® (methylionone), Hedione®, maltol, methyl cedryl ketone, and vanillin.
ethers and acetals, e.g. Ambrox®, geranyl methyl ether, rose oxide or Spirambrene®.
esters and lactones, e.g. benzyl acetate, cedryl actetate, γ-decalactone, Helvetolide®, γ-undecalactone, vetivenyl acetate, cinnamyl propionate, citronellyl acetate, decyl acetate, dimethylbenzylcarbinyl acetate, ethyl acetoacetate, ethyl acetylacetate, cis-3-hexenyl isobutyrate, linalyl acetate and geranyl acetate.
macrocycles, e.g. Ambrettolide, Ethylene brassylate or Exaltolide®.
heterocycles, e.g. isobutylchinoline.

The compounds of the present invention may be used in a broad range of fragrance applications, e.g. in any field of fine and functional perfumery, such as perfumes, household products, laundry products, body care products and cosmetics. The compounds can be employed in widely varying amounts, depending upon the specific application and on the nature and quantity of other odorant ingredients. The proportion is typically from 0.001 to 20 weight percent of the application. In one embodiment, compounds of the present invention may be employed in a fabric softener in an amount of from 0.001 to 0.05 weight percent. In another embodiment, compounds of the present invention may be used in an alcoholic solution in amounts of from 0.1 to 30 weight percent, more preferably between 5 and 20 weight percent. However, these values are given only by way of example, since the experienced perfumer may also achieve effects or may create novel accords with lower or higher concentrations, e.g. up to about 50 weight percent based on the fragrance composition.

The compounds of the present invention may be employed into the fragrance application simply by directly mixing the fragrance composition with the fragrance application, or they may, in an earlier step, be entrapped with an entrapment material such as polymers, capsules, microcapsules and nanocapsules, liposomes, film formers, absorbents such as carbon or zeolites, cyclic oligosaccharides and mixtures thereof, and/or they may be chemically bonded to substrates, which are adapted to release the fragrance molecule upon application of an external stimulus such as light, enzyme, or the like, and then mixed with the application.

Thus, the invention additionally provides a method of manufacturing a fragrance application and consumer products resulting therefrom. The method comprises the incorporation therein of a compound of formula (I) as a fragrance ingredient, either by directly admixing the compound to the application or by admixing a fragrance composition comprising a compound of formula (I) or a precursor thereof, which may then be mixed to a fragrance application, using conventional techniques and methods. Through the addition of an olfactory acceptable amount of a compound of the present invention as hereinabove described, the odor notes of a fragrance application will be improved, enhanced or modified.

By “precursors” is meant, in particular, reaction products of the aldehydes/ketones of formula (I), i.e. compounds of formula (I) wherein R³ and R⁴ together with the carbon atom to which they are attached form a carbonyl group, with a compound comprising at least one functional group selected from the group of primary amine, secondary amine, sulfhydryl (thiol), hydroxyl and carboxyl, in which a covalent bond is formed between at least one carbon atom of the compound of formula (I) and at least one of the hetero atoms (i.e. N, S, and/or O) of said compounds comprising at least one functional group. Thus, the invention furthermore provides a method for improving, enhancing or modifying a fragrance application through the addition thereto of an olfactory acceptable amount of a compound of formula (I), or a mixture thereof.

The invention also provides a fragrance application comprising:
a) as odorant a compound of formula (I) or a mixture thereof; and
b) a consumer product base.

As used herein, “fragrance application” means any products, such as fine fragrances, e.g. eaux de perfume and eaux de toilette; household products, e.g. detergents for dishwasher, surface cleaner, air freshener; laundry products, e.g. softener, bleach, detergent; body care products, e.g. after-shave lotion, shampoo, shower gel, shower and bath salt, hygiene product; and cosmetics, e.g. deodorants, vanishing cremes, comprising an odorant. This list of products is given by way of illustration and is not to be regarded as being in any way limiting.

As used herein, “fragrance composition” means any composition comprising at least one odorant molecule and a diluent conventionally used in conjunction with odorants in fragrance compositions, such as dipropylenglycol (DPG), isopropylmyristate (IMP), trietyhlcitrate (TEC) and alcohol (e.g. ethanol).

As used herein, “consumer product base” means a composition for use as a consumer product to fulfill specific actions, such as cleaning, softening, and caring or the like. Examples of such products include fabric care and personal care products such as laundry care detergents, rinse conditioners, personal cleansing compositions. The composition may comprise a variety of active ingredients such as surfactants, polymers, fillers and auxiliary agents, such as dyes and solvents.

Most of the compounds of formula (I) are described hereinabove for the first time and thus are novel in its own right. To the best of our knowledge only 3 compounds falling under the definition of formula (I) have been described before in literature. All three, namely 4-(2,2,3-trimethylcyclopent-3-enyl)butan-2-one, 4-(2,3,3-trimethylcyclopent-1-enyl)butan-2-one, and 3-methyl-5-(2,3,3-trimethylcyclopent-1-enyl)pent-1-en-3-ol are described by Ribas et al. (Anales de Quimica, Serie C: Quimica Organica y Bioquimica (1982, 78(1), 48-52) as intermediates for the production of the cecropia C₁₇ juvenile hormone analogs. However no odor properties are disclosed.

Accordingly, the present invention refers in a further aspect to compounds of formula (I)

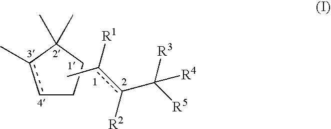

wherein
R¹ and R² are independently hydrogen, or C₁-C₃ alkyl, e.g. ethyl;
C-1 is attached to C-1′ or C-4′;
the dotted line between C-1 and C-2 represents together with the carbon-carbon bond a double bond or a single bond;
the dotted line between C-3′ and C-4′ represents together with the carbon-carbon bond a double bond or a single bond;
I) R³ and R⁴ together with the carbon atom to which they are attached form a carbonyl group; and
   R⁵ is hydrogen, C₁-C₆ alkyl, e.g. ethyl, butyl, or isopropyl, or C₂-C₆ alkenyl, e.g. butenyl or isopropenyl;
   or
II) R³ is hydroxyl; and
   R⁴ and R⁵ are independently from each other hydrogen, C₁-C₆ alkyl, or C₂-C₆ alkenyl; with the proviso that at least one of R¹, R², R⁴ and R⁵ is not hydrogen;
      and the compound of formula (I) comprises up to 20 carbon atoms, preferably 11 to 18, e.g. 12, 13, 14, or 15;
      with the proviso that 4-(2,2,3-trimethylcyclopent-3-enyl)butan-2-one, 4-(2,3,3-trimethylcyclopent-1-enyl)butan-2-one, and 3-methyl-5-(2,3,3-trimethylcyclopent-1-enyl)pent-1-en-3-ol are excluded.

The compounds of formula (I) may be prepared starting from campholytic aldehyde ((S)-(+) campholytic aldehyde or (R)-(−)-campholytic aldehyde) or 2,3,3-trimethylcyclopent-1-enecarbaldehyde. They may be prepared by an aldol-type condensation with the corresponding aldehyde/ketone under acidic or basic conditions or by Wittig-type coupling with a (triphenylphosphoranylidene)alkanone, such as 1-(triphenylphosphoranylidene)-2-propanone or 3-(triphenylphosphoranylidene)-2-butanone, under conditions well known to the person skilled in the art. Whereas under acidic conditions, the corresponding rearranged ([1,2]-methyl shifted) 2-substituted 1,5,5-trimethylcyclopentene-derivatives may be obtained, i.e. compounds of formula (I) wherein the side chain is attached at C-4′, under basic or Wittig-type conditions, the unchanged carbon skeleton of the 4-substituted 1,5,5-trimethylcyclopentene-derivatives is retained, i.e. compounds of formula (I) wherein the side chain is attached to C-1′.

The said rearrangement, namely the [1,2]-methyl shift, can also be effected prior to the condensation step. Thus, the campholytic aldehyde is treated with an acid, such as phosphoric or sulfuric, and the so-obtained 2,3,3-trimethylcyclopent-1-enecarbaldehyde can then be further converted to compounds of formula (I).

The thus-obtained ketones/aldehydes of formula (I) may be reduced with either sodium borohydride or lithium aluminiumhydride, resulting in further compounds of formula (I), namely the corresponding alcohols.

Similarly, the ketone/aldehyde may be converted to the corresponding carbinols by reaction with organometallic species such as Grignard- or organolithium-reagents under conditions well known in the art.

Also, the double bonds between C-3′ and C-4′ in the ring and/or between C-1 and C-2 installed by the above described aldol-condensation or Wittig-type reaction in the side chain may be hydrogenated either together or regioselectively with hydrogen under precious metal catalysis, such as palladium on charcoal under conditions well known in the art.

Further particulars as to reaction conditions are provided in the examples.

The invention is now further described with reference to the following non-limiting examples. These examples are for the purpose of illustration only and it is understood that variations and modifications can be made by one skilled in the art.

Flash chromatography was carried out on Merck silica gel 60 (230-400 mesh). The reported NMR spectra were measured in CDCl₃ if not otherwise stated; chemical shifts (δ) are reported in ppm downfield from TMS; coupling constants J in Hz.

### EXAMPLE 1

### Campholytic Aldehyde

### A) 4-((1E)-2-(2,2,3-Trimethylcyclopent-3-enyl)vinyl)morpholine

A mixture of campholenic aldehyde (138 g, 0.9 mol) and morpholine (95.7 g, 1.1 mol) was dissolved in cyclohexane (400 ml) and a catalytic amount of p-toluenesulfonic acid (1 g) was added. The mixture was heated at reflux with stirring. Under Dean-Stark conditions, approx. 18 g of water was collected within 3 h. Upon cooling to room temperature, the mixture was washed with water (200 ml), dried over sodium sulfate and concentrated in vacuo to furnish the crude morpholine enamine (235 g). This was purified by distillation over a 5 cm-Vigreux column at reduced pressure to afford the title compound (186 g) as a pale yellow oil, boiling point 110° C. (0.1 mbar).

¹H-NMR: δ5.80 (1H, d, J=13.8, ═CHN), 5.25-5.23 (1H, m, ═CH), 4.48 (1H, dd, J=13.8, 9.2, ═CH), 3.73 (4H, apparent t, J=4.9, 2×OCH₂), 2.80 (4H, apparent t, J=4.9, 2×NCH₂), 2.27-2.21 (2H, m, CH and CHH), 2.08-1.97 (1H, m, CHH), 1.62-1.60 (3H, m, Me), 0.92 (3H, s, Me), and 0.74 (3H, s, Me). ¹³C-NMR: δ148.3 (s), 139.8 (d), 121.6 (d), 103.2 (d), 66.5 (2t), 52.6 (d), 49.6 (2t), 47.7 (s), 36.9 (t), 25.1 (q), 20.2 (q), and 12.9 (q). MS: 221 (M⁺, 100), 206 (93), 178 (13), 139 (33), 126 (25), 119 (32), 113 (29), 91 (24), 79 (16), 67 (14), 55 (16), 41 (24).

### B) Campholytic aldehyde (2,2,3-trimethylcyclopent-3-enecarbaldehyde)

4-((1E)-2-(2,2,3-trimethylcyclopent-3-enyl)vinyl)morpholine (45 g, 0.2 mol) was dissolved in acetonitrile (200 ml) and copper(I) chloride (1 g, 0.01 mol) was added. Then, a stream of oxygen is bubbled through the solution for 3.5 h at 25-30° C. and under vigorous stirring, after which the starting morpholine enamine was fully consumed. The mixture was poured into ice/water (500 ml) and extracted with hexane (3×250 ml). The combined organic phases were washed with ammonium chloride solution (aq., sat., 250 ml), dried over sodium sulfate and concentrated in vacuo to give the crude product as a yellowish to greenish liquid (29.6 g). This was distilled under reduced pressure through a 6 cm-Widmer column to afford the required campholytic aldehyde (22.5 g) as a very pale yellow oil, bp. 67-68° C. (˜15 mbar).

¹H-NMR: δ9.76 (1H, d, J=3.2, CHO), 5.26-5.24 (1H, m, ═CH), 2.69-2.65 (1H, m, CH), 2.63-2.55 (1H, m, CHH), 2.41-2.31 (1H, m, CHH), 1.63-1.60 (3H, m, Me), 1.21 (3H, s, Me), and 1.00 (3H, s, Me). ¹³C-NMR: δ204.5 (d), 146.8 (s), 121.0 (d), 61.5 (d), 48.9 (s), 29.3 (t), 27.0 (q), 21.6 (q), and 11.7 (q). MS: 138 (M⁺, 26), 123 (58), 109 (22), 95 (100), 79 (20), 67 (48), 55 (28), 41 (23).

When starting with from (R)-campholenic aldehyde (˜80% ee), the optical rotation is: [α]_{D}²²=+9.8° (5.09 in EtOH). When starting with from (S)-campholenic aldehyde (˜30% ee), the optical rotation is: [α]_{D}²²=−3.8° (5.01 in EtOH).

### EXAMPLE 2

### 4-(2,2,3-Trimethylcyclopent-3-enyl)but-3-en-2-one

A mixture of campholytic aldehyde (from Example 1, 6 g, 0.04 mol) and (triphenylphosphoranylidene)acetone (15 g, 0.04 mol) in diglyme (40 ml) was heated to 170° C. under stirring and kept at this temperature for 15 minutes. The mixture was allowed to cool below 100° C. and poured into water (200 ml), whereby triphenylphosphine oxide precipitates. The mixture was extracted with hexane (3×200 ml) and the combined organic phases were dried over sodium sulfate and concentrated in vacuo (7.4 g). The crude was purified by flash chromatography (MTBE/hexane 1:6) to afford the desired product as a very pale yellow oil (5.8 g, 81%), a mixture of E:Z-isomers (7:2-ratio).

¹H NMR (of the major, E-isomer): δ6.86 (1H, dd, J=16, 8.5, ═CH), 6.09 (1H, dd, J=16, 1, ═CH), 5.26-5.24 (1H, m, ═CH), 2.58 (1H, dq, J=7.5, 1, CH), 2.38-2.31 (1H, m, CHH), 2.27 (3H, s, COMe), 2.25-2.18 (1H, m, CHH), 1.63-1.60 (3H, m, Me), 1.03 (3H, s, Me), and 0.82 (3H, s, Me). ¹³C NMR (of the major, E-isomer): δ198.3 (s), 149.3 (d), 147.7 (s), 131.4 (d), 121.0 (d), 53.7 (d), 49.1 (s), 34.6 (t), 26.9 (q), 25.5 (q), 20.6 (q), 12.4 (q). MS: 178 (M⁺, 13), 163 (24), 145 (26), 135 (43), 121 (28), 107 (28), 105 (31), 96 (44), 95 (41), 93 (55), 91 (37), 81 (33), 79 (28), 77 (27), 55 (20), 43 (100), 41 (28), 39 (10).

When starting with campholytic aldehyde derived from (S)-campholenic aldehyde (˜30% ee), the optical rotation is: [α]_{D}²²=+2.1° (1.12 in CHCl₃).

Odour description: agrestic, woody (thujone-type), sweet, powdery, ionone, floral, creamy.

When starting with campholytic aldehyde derived from (R)-campholenic aldehyde (˜80% ee), the optical rotation is: [α]_{D}²²=−5.5° (1.05 in CHCl₃)

Odour description: floral, ionone alpha, woody, fruity.

### EXAMPLE 3

### (3E)-3-Methyl-4-(2,3,3-trimethylcyclopent-1-enyl)but-3-en-2-one

A mixture of campholytic aldehyde (34.5 g, 0.27 mol), butan-2-one (72 g, 1.0 mol) and Amberlyst® 15 (10 g) was stirred under reflux overnight. After a short path distillation, the crude product (27 g) was purified by flash chromatography (MTBE/hexane 1:19; 12 g, 25% yield, colourless oil).

¹H NMR: δ7.26 (br. s, 1H), 2.67-2.61 (m, 2H), 2.38 (s, 3H), 1.93 (d, J=1.0, 3H), 1.73-1.69 (m, 2H), 1.72 (t, J=2.0, 3H), 1.05 (s, 6H). ¹³C NMR: δ200.5 (s), 154.2 (s), 135.7 (d), 134.7 (s), 131.5 (s), 46.8 (s), 39.1 (t), 32.6 (t), 26.0 (2q), 25.7 (q), 12.6 (q), 10.9 (q). MS: 192 (M⁺, 4), 177 (9), 159 (14), 144 (5), 136 (10), 137 (100), 119 (8), 107 (7), 105 (8), 91 (15), 77 (9), 55 (6), 43 (32), 41 (10).

Odour description: woody, floral, food-like, (methyl)ionone, liquorice, orris, irone, Koavone, greasy, impart creaminess.

### EXAMPLE 4

### (3E)-3-Methyl-4-(2,3,3-trimethylcyclopent-1-enyl)but-3-en-2-ol

Lithium aluminium hydride (0.1 g, 2.6 mmol) was added in five portions to a solution of the ketone from Example 3 (2.0 g, 10 mmol) in THF (20 ml), at 0-10° C. The reaction mixture was allowed to warm up to room temperature and stirring continued for further 0.5 h. Water (0.1 g), 16% solution of sodium hydroxide (0.1 g) and again water (0.3 g) were added successively at 0-10° C. After 15 min. stirring, the solid was filtered off and the filtrate concentrated in vacuo and purified by flash chromatography (MTBE/hexane 1:3) to furnish the desired product (1.5 g) as a colourless oil.

¹H NMR: δ6.07 (br. s, 1H), 4.28 (q, J=6.4, 1H), 2.56-2.41 (m, 2H), 1.75 (br. s, 3H), 1.65 (t, J=7.1, 2H), 1.58-1.55 (m, 3H), 1.29 (d, J=6.4, 3H), 1.00 (s, 6H). ¹³C NMR: δ 145.0 (s), 139.0 (s), 131.0 (s), 120.8 (d), 74.2 (d), 46.3 (s), 39.3 (t), 33.2 (t), 26.3 (q), 26.2 (q), 21.7 (q), 13.2 (q), 10.3 (q). MS: 194 (M⁺, 8), 179 (25), 161 (30), 133 (6), 119 (16), 107 (100), 105 (19), 95 (15), 91 (24), 79 (15), 77 (13), 55 (13), 45 (4), 43 (20).

Odour description: woody, earthy/mossy, floral, honey, Isoraldeine (methylionone), natural, Cetonal.

### EXAMPLE 5

### 2,3,3-Trimethylcyclopent-1-enecarbaldehyde

A mixture of campholytic aldehyde (from Example 1, 10.0 g, 0.72 mol) and Amberlyst® 15 (12 g) and methylcyclohexane (30 ml) was stirred under reflux for 4.5 h. After filtration, the crude product was concentrated in vacuo and purified by bulb-to-bulb distillation to afford the desired aldehyde (4.0 g, 93% GC-purity) as a pale yellow oil.

¹H NMR: δ10.0 (s, 1H), 2.50-2.42 (m, 2H), 2.04 (t, J=1.7, 3H), 1.70 (t, J=7.3, 2H), 1.10 (s, 6H). ¹³C NMR: δ189.2 (d), 168.6 (s), 136.4 (s), 49.2 (s), 37.8 (t), 26.9 (t), 25.5 (2q), 10.0 (q). MS: 138 (M⁺, 32), 123 (32), 95 (100), 92 (9), 79 (10), 77 (13), 67 (32), 55 (18), 53 (13), 41 (15), 39 (14).

### EXAMPLE 6

### Fragrance Composition

| | parts by |
|---|---|
| Compound/Ingredient | weight |
| Patchouli oil | 20 |
| Vetivenyl Acetate | 40 |
| Beta Ionone | 45 |
| Lilial | 75 |
| Peach Pure (gamma-undecalactone) | 15 |
| Tropional (alpha-methyl-1,3-benzodioxole-5-propanal) | 45 |
| Cyclohexal | 20 |
| Ethyl Vanillin | 7 |
| Coriander seed oil | 10 |
| Hydroxycitronellal | 30 |
| Rose oil | 1 |
| Ethyl Linalool | 250 |
| Galbanone (1-(5,5-dimethyl-1-cyclohexen-1-yl)-4-penten-1-one)) | 8 |
| 10% in dipropylene glycol | |
| Methyl Dihydro Isojasmonate | 250 |
| Moxalone (6,7-epoxy-1,1,2,4,4,7-hexamethyl-1,2,3,4,5,6,7,8- | |
| octahydro-naphthalene) 50% in triethyl citrate | 20 |
| Dipropylene Glycol | 134 |

The addition of 30 parts of (S)-(−)-4-(2,2,3-trimethylcyclopent-3-enyl)-but-3-en-2-one to the above composition augments volume, comfort and sensuality. It enhances the fruity animalic side of this Chypre-type fragrance and keeps a soft floral woodyness at the dry down.

1. A flavour or fragrance ingredient comprising a compound of formula (I) 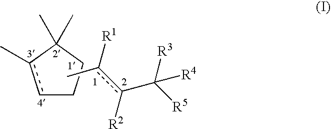
wherein
R¹ and R² are independently hydrogen, or C₁-C₃ alkyl;
C-1 is attached to C-1′ or C-4′;
the dotted line between C-1 and C-2 represents together with the carbon-carbon bond a double bond or a single bond;
the dotted line between C-3′ and C-4′ represents together with the carbon-carbon bond a double bond or a single bond;
I) R³ and R⁴ together with the carbon atom to which they are attached form a carbonyl group; and
R⁵ is hydrogen, C₁-C₆ alkyl, or C₂-C₆ alkenyl;
or
II) R³ is hydroxyl; and
R⁴ and R⁵ are independently from each other hydrogen, C₁-C₆ alkyl, or C₂-C₆ alkenyl;
with the proviso that at least one of R¹, R², R⁴ and R⁵ is not hydrogen;
and the compound of formula (I) comprises up to 20 carbon atoms.

2. A flavour or fragrance ingredient according to claim 1 wherein the relative configuration of the ring system of the compound of formula (I) is 1′R.

3. A flavour or fragrance ingredient according to claim 1 wherein the relative configuration of the ring system of the compound of formula (I) is 1′S.

4. A flavour or fragrance ingredient according to claim 1 wherein the compound of formula (I) is selected from the list consisting of 4-(2,2,3-trimethylcyclopent-3-enyl)but-3-en-2-one, (3E)-3-methyl-4-(2,3,3-trimethylcyclopent-1-enyl)but-3-en-2-one and (3E)-3-methyl-4-(2,3,3-trimethylcyclopent-1-enyl)but-3-en-2-ol.

5. (canceled)

6. A fragrance application comprising
b) a compound of formula (I) according to claim 1; and
b) a consumer product base.

7. A fragrance application according to claim 6 wherein the consumer product base is selected from the group consisting of fine fragrance, household product, laundry product, body care product and cosmetic.

8. A method of manufacturing a flavour or fragrance composition, comprising the step of:
incorporating an effective amount of a compound of formula (I), or a mixture thereof to a base material.

9. A compound of formula (I) 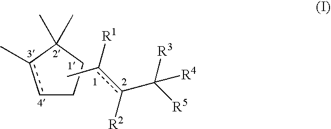
wherein
R¹ and R² are independently hydrogen, or C₁-C₃ alkyl;
C-1 is attached to C-1′ or C-4′;
the dotted line between C-1 and C-2 represents together with the carbon-carbon bond a double bond or a single bond;
the dotted line between C-3′ and C-4′ represents together with the carbon-carbon bond a double bond or a single bond;
I) R³ and R⁴ together with the carbon atom to which they are attached form a carbonyl group; and
R⁵ is hydrogen, C₁-C₆ alkyl, or C₂-C₆ alkenyl;
or
II) R³ is hydroxyl; and
R⁴ and R⁵ are independently from each other hydrogen, C₁-C₆ alkyl, or C₂-C₆ alkenyl;
with the proviso that at least one of R¹, R², R⁴ and R⁵ is not hydrogen;
and the compound of formula (I) comprises up to 20 carbon atoms; with the proviso that 4-(2,2,3-trimethylcyclopent-3-enyl)butan-2-one, 4-(2,3,3-trimethylcyclopent-1-enyl)butan-2-one, and 3-methyl-5-(2,3,3-trimethylcyclopent-1-enyl)pent-1-en-3-ol are excluded.

10. A compound according to claim 9 selected from the list consisting of:
4-(2,2,3-trimethylcyclopent-3-enyl)but-3-en-2-one,
(3E)-3-methyl-4-(2,3,3-trimethylcyclopent-1-enyl)but-3-en-2-one and
(3E)-3-methyl-4-(2,3,3-trimethylcyclopent-1-enyl)but-3-en-2-ol.
